SEQUENCE LISTING (77 sequences)

SEQ ID NO: 1 - protein, 10 aa, synthetic construct
  REGION 1..10: note = Foot protein 1 decapeptide repeat
AKPSYPPTYK

SEQ ID NO: 2 - protein, 52 aa, synthetic construct
  REGION 1..52: note = Foot protein type 3 (FP-3, Mytilus edulis)
ADYYGPKYGPPRRYGGGNYNRYGGSRRYGGYKGWNNGWKRGRWGRKYYEFEF

SEQ ID NO: 3 - protein, 75 aa, synthetic construct
  REGION 1..75: note = Foot protein type 5 (FP-5, Mytilus edulis)
SSEEYKGGYYPGNAYHYHSGGSYHGSGYHGGYKGKYYGKAKKYYYKYKNSGKYKYLKKARKYHRKGYKKYYGGSS

SEQ ID NO: 4 - protein, 76 aa, synthetic construct
  REGION 1..76: note = Foot protein type 5 (FP-5, Mytilus galloprovincialis)
SSEEYKGGYYPGNTYHYHSGGSYHGSGYHGGYKGKYYGKAKKYYYKYKNSGKYKYLKKARKYHRKGYKKYYGGGSS

SEQ ID NO: 5 - protein, 194 aa, synthetic construct
  REGION 1..194: note = Hybrid mussel adhesive protein (FP-151, MEFP-5 based: Kollodis)
AKPSYPPTYKAKPSYPPTYKAKPSYPPTYKAKPSYPPTYKAKPSYPPTYKAKPSYPPTYKSSEEYKGGYYPGNAYHYHSGGSYHGSGYHGGYKGKYYGKAKKYYYKYKNSGKYKYLKKARKYHRKGYKYYGGSSAKPSYPPTYKAKPSYPPTYKAKPSYPPTYKAKPSYPPTYKAKPSYPPTYKAKPSYPPTYK

SEQ ID NO: 6 - protein, 196 aa, synthetic construct
  REGION 1..196: note = Hybrid mussel adhesive protein (FP-151, MGFP-5 based)
AKPSYPPTYKAKPSYPPTYKAKPSYPPTYKAKPSYPPTYKAKPSYPPTYKAKPSYPPTYKSSEEYKGGYYPGNTYHYHSGGSYHGSGYHGGYKGKYYGKAKKYYYKYKNSGKYKYLKKARKYHRKGYKKYYGGGSSAKPSYPPTYKAKPSYPPTYKAKPSYPPTYKAKPSYPPTYKAKPSYPPTYKAKPSYPPTYK

SEQ ID NO: 7 - protein, 11 aa, synthetic construct
  REGION 1..11: note = Fibronectin I-derived peptide (KYILRWRPKNS)
KYILRWRPKNS

SEQ ID NO: 8 - protein, 16 aa, synthetic construct
  REGION 1..16: note = Fibronectin III-derived peptide (YRVRVTPKEKTGPMKE)
YRVRVTPKEKTGPMKE

SEQ ID NO: 9 - protein, 9 aa, synthetic construct
  REGION 1..9: note = Fibronectin III-derived peptide (SPPRRARVT)
SPPRRARVT

SEQ ID NO: 10 - protein, 9 aa, synthetic construct
  REGION 1..9: note = Fibronectin III-derived peptide (ATETTITIS)
ATETTITIS

SEQ ID NO: 11 - protein, 32 aa, synthetic construct
  REGION 1..32: note = Fibronectin III-derived peptide (VSPPRRARVTDATETTITISWRTKTETITGFG)
VSPPRRARVTDATETTITISWRTKTETITGFG

SEQ ID NO: 12 - protein, 12 aa, synthetic construct
  REGION 1..12: note = Fibronectin III-derived peptide (ANGQTPIQRYIK)
ANGQTPIQRYIK

SEQ ID NO: 13 - protein, 11 aa, synthetic construct
  REGION 1..11: note = Fibronectin III-derived peptide (KPDVRSYTITG)
KPDVRSYTITG

SEQ ID NO: 14 - protein, 30 aa, synthetic construct
  REGION 1..30: note = Fibronectin-derived peptide (PRARITGYIIKYEKPGSPPREVVPRPRPGV)
PRARITGYIIKYEKPGSPPREVVPRPRPGV

SEQ ID NO: 15 - protein, 8 aa, synthetic construct
  REGION 1..8: note = Fibronectin III-derived peptide (WQPPRARI)
WQPPRARI

SEQ ID NO: 16 - protein, 19 aa, synthetic construct
  REGION 1..19: note = Fibronectin III-derived peptide (WQPPRARITGYIIKYEKPG)
WQPPRARITGYIIKYEKPG

SEQ ID NO: 17 - protein, 17 aa, synthetic construct
  REGION 1..17: note = Fibronectin III-derived peptide (YEKPGSPPREVVPRPRP)
YEKPGSPPREVVPRPRP

SEQ ID NO: 18 - protein, 15 aa, synthetic construct
  REGION 1..15: note = Fibronectin III-derived peptide (KNNQKSEPLIGRKKT)
KNNQKSEPLIGRKKT

SEQ ID NO: 19 - protein, 12 aa, synthetic construct
  REGION 1..12: note = Laminin alpha1-derived peptide (GLIYYVAHQNQM)
GLIYYVAHQNQM

SEQ ID NO: 20 - protein, 12 aa, synthetic construct
  REGION 1..12: note = Laminin alpha1-derived peptide (RKRLQVQLSIRT)
RKRLQVQLSIRT

SEQ ID NO: 21 - protein, 12 aa, synthetic construct
  REGION 1..12: note = Laminin alpha2-derived peptide (GLLFYMARINHA)
GLLFYMARINHA

SEQ ID NO: 22 - protein, 12 aa, synthetic construct
  REGION 1..12: note = Laminin alpha3-derived peptide (KNSFMALYLSKG)
KNSFMALYLSKG

SEQ ID NO: 23 - protein, 12 aa, synthetic construct
  REGION 1..12: note = Laminin alpha4-derived peptide (VVRDITRRGKPG)
VVRDITRRGKPG

SEQ ID NO: 24 - protein, 12 aa, synthetic construct
  REGION 1..12: note = Laminin alpha4-derived peptide (RAYFNGQSFIAS)
RAYFNGQSFIAS

SEQ ID NO: 25 - protein, 12 aa, synthetic construct
  REGION 1..12: note = Laminin alpha4-derived peptide (GEKSQFSIRLKT)
GEKSQFSIRLKT

SEQ ID NO: 26 - protein, 20 aa, synthetic construct
  REGION 1..20: note = Laminin alpha4-derived peptide (TLFLAHGRLVFMFNVGHKKL)
TLFLAHGRLVFMFNVGHKKL

SEQ ID NO: 27 - protein, 12 aa, synthetic construct
  REGION 1..12: note = Laminin alpha4-derived peptide (TLFLAHGRLVFM)
TLFLAHGRLVFM

SEQ ID NO: 28 - protein, 12 aa, synthetic construct
  REGION 1..12: note = Laminin alpha4-derived peptide (LVFMFNVGHKKL)
LVFMFNVGHKKL

SEQ ID NO: 29 - protein, 12 aa, synthetic construct
  REGION 1..12: note = Laminin alpha4-derived peptide (GAAWKIKGPIYL)
GAAWKIKGPIYL

SEQ ID NO: 30 - protein, 12 aa, synthetic construct
  REGION 1..12: note = Laminin alpha4-derived peptide (VIRDSNVVQLDV)
VIRDSNVVQLDV

SEQ ID NO: 31 - protein, 12 aa, synthetic construct
  REGION 1..12: note = Laminin alpha5-derived peptide (GKNTGDHFVLYM)
GKNTGDHFVLYM

SEQ ID NO: 32 - protein, 13 aa, synthetic construct
  REGION 1..13: note = Laminin alpha5-derived peptide (RLVSYSGVLFFLK)
RLVSYSGVLFFLK

SEQ ID NO: 33 - protein, 12 aa, synthetic construct
  REGION 1..12: note = Laminin alpha5-derived peptide (GPLPSYLQFVGI)
GPLPSYLQFVGI

SEQ ID NO: 34 - protein, 12 aa, synthetic construct
  REGION 1..12: note = Laminin alpha5-derived peptide (RNRLHLSMLVRP)
RNRLHLSMLVRP

SEQ ID NO: 35 - protein, 12 aa, synthetic construct
  REGION 1..12: note = Laminin alpha5-derived peptide (LVLFLNHGHFVA)
LVLFLNHGHFVA

SEQ ID NO: 36 - protein, 12 aa, synthetic construct
  REGION 1..12: note = Laminin alpha5-derived peptide (AGQWHRVSVRWG)
AGQWHRVSVRWG

SEQ ID NO: 37 - protein, 12 aa, synthetic construct
  REGION 1..12: note = Laminin alpha5-derived peptide (KMPYVSLELEMR)
KMPYVSLELEMR

SEQ ID NO: 38 - protein, 7 aa, synthetic construct
  REGION 1..7: note = Laminin alpha5-derived peptide (RYVVLPR)
RYVVLPR

SEQ ID NO: 39 - protein, 12 aa, synthetic construct
  REGION 1..12: note = Laminin-derived peptide (VRWGMQQIQLVV)
VRWGMQQIQLVV

SEQ ID NO: 40 - protein, 12 aa, synthetic construct
  REGION 1..12: note = Laminin-derived peptide (TVFSVDQDNMLE)
TVFSVDQDNMLE

SEQ ID NO: 41 - protein, 13 aa, synthetic construct
  REGION 1..13: note = Laminin-derived peptide (APMSGRSPSLVLK)
APMSGRSPSLVLK

SEQ ID NO: 42 - protein, 12 aa, synthetic construct
  REGION 1..12: note = Laminin-derived peptide (VLVRVERATVFS)
VLVRVERATVFS

SEQ ID NO: 43 - protein, 10 aa, synthetic construct
  REGION 1..10: note = Laminin-derived peptide (RNIAEIIKDI)
RNIAEIIKDI

SEQ ID NO: 44 - protein, 6 aa, synthetic construct
  REGION 1..6: note = Collagen I-derived peptide (KGHRGF)
KGHRGF

SEQ ID NO: 45 - protein, 12 aa, synthetic construct
  REGION 1..12: note = Collagen IV-derived peptide (TAGSCLRKFSTM)
TAGSCLRKFSTM

SEQ ID NO: 46 - protein, 13 aa, synthetic construct
  REGION 1..13: note = Collagen IV-derived peptide (GEFYFDLRLKGDK)
GEFYFDLRLKGDK

SEQ ID NO: 47 - protein, 16 aa, synthetic construct
  REGION 1..16: note = Vitronectin-derived peptide (KKQRFRHRNRKGYRSQ)
KKQRFRHRNRKGYRSQ

SEQ ID NO: 48 - protein, 4 aa, synthetic construct
  REGION 1..4: note = Bone sialoprotein-derived peptide (KRSR)
KRSR

SEQ ID NO: 49 - protein, 4 aa, synthetic construct
  REGION 1..4: note = Bone sialoprotein-derived peptide (KRRA)
KRRA

SEQ ID NO: 50 - skipped

SEQ ID NO: 51 - protein, 4 aa, synthetic construct
  REGION 1..4: note = Vitronectin-derived peptide (RGDV)
RGDV

SEQ ID NO: 52 - protein, 12 aa, synthetic construct
  REGION 1..12: note = Vitronectin-derived peptide (PQVTRGDVFTMP)
PQVTRGDVFTMP

SEQ ID NO: 53 - protein, 6 aa, synthetic construct
  REGION 1..6: note = Fibronectin-derived peptide (GRGDSP)
GRGDSP

SEQ ID NO: 54 - protein, 20 aa, synthetic construct
  REGION 1..20: note = Fibronectin-derived peptide (PHSRNSGSGSGSGSGRGDSP)
PHSRNSGSGSGSGSGRGDSP

SEQ ID NO: 55 - protein, 7 aa, synthetic construct
  REGION 1..7: note = Fibronectin-derived peptide (EDGIHEL)
EDGIHEL

SEQ ID NO: 56 - protein, 9 aa, synthetic construct
  REGION 1..9: note = Tenascin-derived peptide (VAEIDGIEL)
VAEIDGIEL

SEQ ID NO: 57 - protein, 8 aa, synthetic construct
  REGION 1..8: note = Tenascin-derived peptide (VFDNFVLK)
VFDNFVLK

SEQ ID NO: 58 - protein, 25 aa, synthetic construct
  REGION 1..25: note = Fibronectin domain III mimetic (RGDSGSGSGSGSGANGQTPIQRYIK)
RGDSGSGSGSGSGANGQTPIQRYIK

SEQ ID NO: 59 - protein, 224 aa, synthetic construct
  REGION 1..224: note = Fibronectin domain III mimetic (ANGQTPIQRYIK - FP151 - RGD)
ANGQTPIQRYIKAKPSYPPTYKAKPSYPPTYKAKPSYPPTYKAKPSYPPTYKAKPSYPPTYKAKPSYPPTYKSSEEYKGGYYPGNAYHYHSGGSYHGSGYHGGYKGKYYGKAKKYYYKYKNSGKYKYLKKARKYHRKGYKYYGGSSAKPSYPPTYKAKPSYPPTYKAKPSYPPTYKAKPSYPPTYKAKPSYPPTYKAKPSYPPTYKRGDSGSGSGSGSGIDAPS

SEQ ID NO: 60 - protein, 20 aa, synthetic construct
  REGION 1..20: note = 2 times repeated sequence derived from foot protein 1 (FP-1)
AKPSYPPTYKAKPSYPPTYK

SEQ ID NO: 61 - protein, 60 aa, synthetic construct
  REGION 1..60: note = 6 times repeated sequence derived from foot protein 1 (FP-1)
AKPSYPPTYKAKPSYPPTYKAKPSYPPTYKAKPSYPPTYKAKPSYPPTYKAKPSYPPTYK

SEQ ID NO: 62 - protein, 39 aa, synthetic construct
  REGION 1..39: note = Partial sequence of foot protein type 2 (FP-2)
EVHACKPNPCKNNGRCYPDGKTGYKCKCVGGYSGPTCAC

SEQ ID NO: 63 - protein, 46 aa, synthetic construct
  REGION 1..46: note = Foot protein type 3 (FP-3, Mytilus galloprovincialis: mgfp-3A)
ADYYGPKYGPPRRYGGGNYNRYGRRYGGYKGWNNGWKRGRWGRKYY

SEQ ID NO: 64 - protein, 50 aa, synthetic construct
  REGION 1..50: note = Foot protein type 3 (FP-3, Mytilus edulis: mefp-3F)
ADYYGPNYGPPRRYGGGNYNRYNGYGGGRRYGGYKGWNNGWNRGRRGKYW

SEQ ID NO: 65 - protein, 44 aa, synthetic construct
  REGION 1..44: note = Foot protein type 3 (FP-3, Mytilus californianus)
GAYKGPNYNYPWRYGGKYNGYKGYPRGYGWNKGWNKGRWGRKYY

SEQ ID NO: 66 - protein, 60 aa, synthetic construct
  REGION 1..60: note = Partial sequence from foot protein type 4 (Mytilus californianus)
GHVHRHRVLHKHVHNHRVLHKHLHKHQVLHGHVHRHQVLHKHVHNHRVLHKHLHKHQVLH

SEQ ID NO: 67 - protein, 76 aa, synthetic construct; identical to SEQ ID NO: 4
  REGION 1..76: note = Foot protein 5 (FP-5, Mytilus edulis)

SEQ ID NO: 68 - protein, 71 aa, synthetic construct
  REGION 1..71: note = Foot protein 5 (FP-5, Mytilus coruscus)
YDDYSDGYYPGSAYNYPSGSHWHGHGYKGKYYGKGKKYYYKFKRTGKYKYLKKARKYHRKGYKKHYGGSSS

SEQ ID NO: 69 - protein, 99 aa, synthetic construct
  REGION 1..99: note = Mussel adhesive protein foot protein type 6
GGGNYRGYCSNKGCRSGYIFYDNRGFCKYGSSSYKYDCGNYAGCCLPRNPYGRVKYYCTKKYSCPDDFYYYNNKGYYYYNDKDYFNCGSYNGCCLRSGY

SEQ ID NO: 70 - protein, 192 aa, synthetic construct
  REGION 1..192: note = Hybrid mussel adhesive protein (FP-151, MCFP-5 based)
AKPSYPPTYKAKPSYPPTYKAKPSYPPTYKAKPSYPPTYKAKPSYPPTYKAKPSYPPTYKYDGYSDGYYPGSAYNYPSGSHGYHGHGYKGKYYGKGKKYYYKYKRTGKYKYLKKARKYHRKGYKKYYGGGSSAKPSYPPTYKAKPSYPPTYKAKPSYPPTYKAKPSYPPTYKAKPSYPPTYKAKPSYPPTYK

SEQ ID NO: 71 - protein, 177 aa, synthetic construct
  REGION 1..177: note = Hybrid mussel adhesive protein (FP-131)
AKPSYPPTYKAKPSYPPTYKAKPSYPPTYKAKPSYPPTYKAKPSYPPTYKAKPSYPPTYKGCRADYYGPKYGPPRRYGGGNYNRYGGSRRYGGYKGWNNGWKRGRWGRKYYEFEFAKPSYPPTYKAKPSYPPTYKAKPSYPPTYKAKPSYPPTYKAKPSYPPTYKAKPSYPPTYKKL

SEQ ID NO: 72 - protein, 180 aa, synthetic construct
  REGION 1..180: note = Hybrid mussel adhesive protein (FP-251)
MEVHACKPNPCKNNGRCYPDGKTGYKCKCVGGYSGPTCACSSEEYKGGYYPGNSNHYHSGGSYHGSGYHGGYKGKYYGKAKKYYYKYKNSGKYKYLKKARKYHRKGYKKYYGGSSEFEFAKPSYPPTYKAKPSYPPTYKAKPSYPPTYKAKPSYPPTYKAKPSYPPTYKAKPSYPPTYKK

SEQ ID NO: 73 - protein, 182 aa, synthetic construct
  REGION 1..182: note = Hybrid mussel adhesive protein (FP-353)
GCRADYYGPKYGPPRRYGGGNYNRYGGSRRYGGYKGWNNGWKRGRWGRKYYEFEFYDGYSDGYYPGSAYNYPSGSHGYHGHGYKGKYYGKGKKYYYKYKRTGKYKYLKKARKYHRKGYKKYYGGGSSGCRADYYGPKYGPPRRYGGGNYNRYGGSRRYGGYKGWNNGWKRGRWGRKYYEFEF

SEQ ID NO: 74 - protein, 309 aa, synthetic construct
  REGION 1..309: note = Hybrid mussel adhesive protein (FP-13151)
AKPSYPPTYKAKPSYPPTYKAKPSYPPTYKAKPSYPPTYKAKPSYPPTYKAKPSYPPTYKGCRADYYGPKYGPPRRYGGGNYNRYGGSRRYGGYKGWNNGWKRGRWGRKYYEFEFAKPSYPPTYKAKPSYPPTYKAKPSYPPTYKAKPSYPPTYKAKPSYPPTYKAKPSYPPTYKKLYDGYSDGYYPGSAYNYPSGSHGYHGHGYKGKYYGKGKKYYYKYKRTGKYKYLKKARKYHRKGYKKYYGGGSSAKPSYPPTYKAKPSYPPTYKAKPSYPPTYKAKPSYPPTYKAKPSYPPTYKAKPSYPPTYK

SEQ ID NO: 75 - protein, 223 aa, synthetic construct
  REGION 1..223: note = Fibronectin domain III mimetic (RGD - FP151 - ANGQTPIQRYIK)
RGDSGSGSGSGSGIDAPSAKPSYPPTYKAKPSYPPTYKAKPSYPPTYKAKPSYPPTYKAKPSYPPTYKAKPSYPPTYKSSEEYKGGYYPGNAYHYHSGGSYHGSGYHGGYKGKYYGKAKKYYYKYKNSGKYKYLKKARKYHRKGYKYYGGSSAKPSYPPTYKAKPSYPPTYKAKPSYPPTYKAKPSYPPTYKAKPSYPPTYKAKPSYPPTYANGQTPIQRYIK

SEQ ID NO: 76 - protein, 12 aa, synthetic construct
  REGION 1..12: note = Laminin mimetic (PGRWHKVSVRWE)
PGRWHKVSVRWE

SEQ ID NO: 77 - protein, 12 aa, synthetic construct
  REGION 1..12: note = Laminin mimetic (VLLQANDGAGEF)
VLLQANDGAGEF

(19) United States
(12) Patent Application Publication
(10) Pub. No.: US 2025/0066720 A1
(43) Pub. Date: Feb. 27, 2025

(54) GF BINDING ECM MIMETIC COATED MICROCARRIER

(71) Applicant: STELO BIOTECHNOLOGY CO., LTD., Incheon (KR)

(72) Inventors: Sang Jae LEE, Seoul (KR); Bong Jin HONG, Pohang-si, Gyeongsangbuk-do (KR); Min Chul PARK, Seongnam-si, Gyeonggi-do (KR)

(73) Assignee: STELO BIOTECHNOLOGY CO., LTD., Incheon (KR)

(21) Appl. No.: 18/723,708

(22) PCT Filed: Feb. 21, 2023
(86) PCT No.: PCT/KR2023/002456

§ 371 Date: Jun. 24, 2024

Related U.S. Application Data
(60) Provisional application No. 63/268,303, filed on Feb. 21, 2022.

(51) Int. Cl. C12N 5/00

(52) U.S. Cl. CPC C12N 5/0075; C12N 2531/00; C12N 2533/50; C12N 2533/54; C12N 2533/90

(57) ABSTRACT

Provided are an ECM mimetic microcarrier comprising a positively charged recombinant adhesive protein genetically functionalized with a heparin binding motif which is capable of binding or sequestering growth factors and a negatively charged component, wherein the ECM mimetic microcarrier may be in the form of a mussel adhesive protein where extracellular matrix (ECM) protein derived integrin binding peptide motifs and GF binding peptide motifs are recombinantly incorporated into the mussel adhesive protein, and a use of the ECM mimetic microcarrier for stem cell expansion application.

### CROSS REFERENCE TO RELATED APPLICATIONS

This application claims the benefit of U.S. Provisional Application No. 63/268,303, filed on Feb. 21, 2022, the contents of which are incorporated herein by reference in their entirety.

### TECHNICAL FIELD

### Field of the Invention

The present invention relates to an extracellular matrix (ECM) mimetic-coated microcarrier having growth factor binding peptide motif and method of making and using thereof. In particular embodiments, the present invention is directly related to an ECM mimetic microcarrier comprising one of FGF, TGFβ, PDGF, and VEGF binding peptide motifs for stem cell or stem cell-like primary cell expansion.

### Background Art

Microcarriers in stirred-tank bioreactors have been widely utilized in large-scale expansion of stem cells such as hMSCs for therapeutic applications because of a high surface-to-volume ratio compared to conventional 2D planar culture. The advantages of microcarrier culture in stirred-tank bioreactors include the scalable design, even cell distribution, homogeneous nutrition and oxygen access, and the timely assessment of medium composition and evaluation of cell properties. (Ang-Chen Tsai, et al, Front. Bioeng. Biotechnol., 24 June 2020. Influence of Microenvironment on Mesenchymal Stem Cell Therapeutic Potency: From Planar Culture to Microcarriers).

Nevertheless, recent studies have shown that mesenchymal stem cells cultured on microcarriers differ from those on dishes or flasks in size, morphology, proliferation, viability, surface marker, gene expression, differentiation capacity, and secretion of cytokines, which may lead to the alteration of their therapeutic potency (Hupfeld, J., et al. (2014). Modulation of mesenchymal stromal cell characteristics by microcarrier culture in bioreactors. Biotechnol. Bioeng. 111, 2290-2302). The deviations of cultured cells result from the different microenvironment between 2D planar and microcarrier cultures as well as the change of adhesion surface geography and flow-induced dynamic environment (Ma, T., et al. (2016). Biomanufacturing of human mesenchymal stem cells in cell therapy: influence of microenvironment on scalable expansion in bioreactors. Biochem. Eng. J. 108, 44-50).

If in vivo like microenvironment can be mimicked in vitro, it is possible to manipulate cell behaviors such as cell survival, shape, migration, proliferation, and differentiation, thus leading to the morphology and physiology similar to those in vivo. (Hussey, G. S., et al., Extracellular Matrix-Based Materials for Regenerative Medicine. Nat. Rev. Mater. 2018, 3 (7), 159-173). This mimicry can be generated with culture-substrates made of ECM proteins and growth factors (Julien Nicolas, et al., 3D Extracellular Matrix Mimics: Fundamental Concepts and Role of Materials Chemistry to Influence Stem Cell Fate. Biomacromolecules 2020, 21, 6, 1968-1994).

Growth factors (GFs) and extracellular matrix (ECM) are major components of extracellular microenvironment. Various engineering approaches with these major components have been developed to achieve a higher degree of control over cellular activities and behaviors. In addition, cell signaling occurs when adhesion molecules such as integrin ligands and cell surface growth factor receptors are present in combination with the ECM to create optimum environment (Kim, S.H, et al., Extracellular matrix and cell signalling: The dynamic cooperation of integrin, proteoglycan and growth factor receptor. J. Endocrinol. 2011, 209, 139-151).

Therefore, when cells are cultured on microcarriers in suspension culture system for their mass production, incorporation of these major components to the microcarrier surfaces could enhance cell viability and proliferation.

GFs are involved in the regulation of a variety of cellular processes and typically act as signaling molecules between cells. They promote cell proliferation, differentiation, and maturation. However, such an ability is limited by their poor stability due to degradation and proteolysis under physiological conditions. Hence, they should be delivered periodically which requires some form of continuous delivery for effective therapeutic application. (See R. Pearlman and Y. J. Wang, Formulation, Characterization, and Stability of Protein Drugs: Case Histories, Springer US, Boston, MA, 1st edn, 2002, vol. 9).

To improve the stability of growth factors, marine exopolysaccharide (EPS) microcarriers encapsulating transforming growth factor-β1 (TGF-β1) were developed for cartilage engineering. The release study showed that encapsulated growth factors were released slowly from the microcarriers, which enables the majority of TGF-β1 to be retained inside the microcarrier. Such slow release could make the growth factor available in the target location long enough to induce robust regenerative responses. (Agata Zykwinska, et al, Microcarriers Based on Glycosaminoglycan-Like Marine Exopolysaccharide for TGF-β1 Long-Term Protection. Mar. Drugs 2019, 17 (1), 65).

Soft biohybrid hydrogels containing glycosaminoglycans (GAGs) have been extensively used in order to biomimetically emulate ECM functions (See U. Freudenberg, Y. Liang, K. L. Kiick and C. Werner, Adv. Mater., 2016, 28, 8861-8891). Through their negative charge, sulfated GAGs such as heparin can interact with various growth factors, chemokines, and cytokines. Electrostatic interaction between positively charged sites of protein's surface and the negatively charged GAGs enables both of them to form a complex of which shows enhanced stability against either proteolytic or thermal degradation. Incorporation of heparin into hydrogel networks, therefore, becomes a highly attractive strategy for the loading and sustained delivery of therapeutic proteins. (O. Jeon, C. Powell, L. D. Solorio, M. D. Krebs and E. Alsberg, J. Controlled Release, 2011, 154, 258-266., U. Freudenberg, A. Zieris, K. Chwalek, M. V. Tsurkan, M. F. Maitz, P. Atallah, K. R. Levental, S. A. Eming and C. Werner, J. Controlled Release, 2015, 220, 79-88., S. T. Koshy, D. K. Y. Zhang, J. M. Grolman, A. G. Stafford and D. J. Mooney, Acta Biomater., 2018, 65, 36-43). For example, Lucas and co-workers demonstrated heparin-based cryogel microcarrier load high amount of IL-13 and it releases IL-13 slowly without its functional loss. (Lucas Schirmer, et al., Heparin-based, injectable microcarriers for controlled delivery of interleukin-13 to the brain. Biomater. Sci., 2020, 8, 4997).

In addition to GAGs, extracellular matrix proteins can also be used to stabilize various growth factors. Many extracellular ECM proteins have been known to have binding sites for both growth factors and cell adhesion allowing growth factors to be released locally and efficiently bind to their cell surface receptors. Thus, the ECM functions as a cofactor and enables the growth factor to be exposed to cell surface receptors for a long time. Further, localization of growth factors by ECM binding establishes the concentration gradient of soluble chemokines and growth factor, which play an essential role in developmental processes. Growth factors can also be sequestered to the ECM, which hereby function as a localized reservoir. Previously, we developed protein substrate having ECM mimetic peptide motif to capture various growth factors, allowing their long lasting activity (See PCT/KR2021/019159).

ECM Mimetic protein-based microcarriers are attractive substrate for stem cell expansion. Moreover, ECM mimetic-coated microcarriers with cell-specific ligands allows for active targeting abilities.

The purpose of the present invention is to provides simpler and more reliable ECM mimetic-microcarriers presenting GF complex, as presented in FIG. 1, to deliver GFs with long-lasting stability and functionality for stem cell expansion, by utilizing ECM-derived GF binding peptide motif as well as integrin binding motif.

### DISCLOSURE OF INVENTION

### Technical Problem

An object of the present invention is to provide an ECM mimetic microcarrier comprising a positively charged recombinant adhesive protein genetically functionalized with a growth factor binding motif which is capable of binding or sequestering growth factors and a negatively charged biomolecule such as alginate to form a microcarrier by being mixed with the said recombinant adhesive protein functionalized with growth factor binding motif.

Another object of the present invention is to provide a GF carrier to regulate cell fate, wherein said GF carrier comprises the protein substrate having growth factor binding motif to deliver a GF that can induce growth factor signaling via activating growth factor receptors.

### Solution of Problem

To achieve the objects, in an aspect, the present invention provides an ECM mimetic microcarrier comprising an ECM mimetic protein component genetically functionalized with a growth factor or syndecan binding motif which is capable of binding or sequestering growth factors and a negatively charged component.

In one embodiment, the ECM mimetic protein dominates on surface of the microcarrier as shell substrate and the negatively charged component (for examples, heparin, heparin analogs, low molecular weight heparin, glycosaminoglycans, or alginate) as core substrate. In another embodiment, a substantial portion of the surface of the microcarrier is characterized with a positive surface charge. In a further embodiment, the microcarrier of the present invention comprise at least one positively charged ECM mimetic substrate, wherein the ECM mimetic substrate is selected from any recombinant mussel adhesive protein genetically functionalized with an ECM derived peptide motif, and at least one negatively charged core substrate, wherein the core substrate is selected from the group consisting of heparin, heparin analogs, low molecular weight heparin, glycosaminoglycans, and alginate. In one embodiment, an ECM mimetic protein contains growth factor binding motif to bind or sequester growth factors.

In an embodiment of the present invention, said growth factor binding motif can be derived from fibronectin domain III, laminin globular domain, heparin binding domain of collagen, heparin binding domain of vitronectin, heparin binding domain of fibrinogen or heparin binding domain of bone sialoprotein.

In a preferred embodiment of the present invention, said growth factor binding motif derived from fibronectin domain III can be a peptide of KYILRWRPKNS (SEQ ID NO: 7), YRVRVTPKEKTGPMKE (SEQ ID NO: 8), SPPRRARVT (SEQ ID NO: 9), ATETTITIS (SEQ ID NO: 10), VSPPRRARVTDATETTITISWRTKTETITGFG (SEQ ID NO: 11), ANGQTPIQRYIK (SEQ ID NO: 12), KPDVRSYTITG (SEQ ID NO: 13), PRARITGYIIKYEKPGSPPREVVPRPRPGV (SEQ ID NO: 14), WQPPRARI (SEQ ID NO: 15), WQPPRARITGYIIKYEKPG (SEQ ID NO: 16), YEKPGSPPREVVPRPRP (SEQ ID NO: 17), or KNNQKSEPLIGRKKT (SEQ ID NO: 18).

In another preferred embodiment of the present invention, said growth factor binding motif derived from laminin globular domain can be a peptide of GLIYYVAHQNQM (SEQ ID NO: 19), RKRLQVQLSIRT (SEQ ID NO: 20), GLLFYMARINHA (SEQ ID NO: 21), KNSFMALYLSKG (SEQ ID NO: 22), VVRDITRRGKPG (SEQ ID NO: 23), RAYFNGQSFIAS (SEQ ID NO: 24), GEKSQFSIRLKT (SEQ ID NO: 25), TLFLAHGRLVFMFNVGHKKL (SEQ ID NO: 26), TLFLAHGRLVFM (SEQ ID NO: 27), LVFMFNVGHKKL (SEQ ID NO: 28), GAAWKIKGPIYL (SEQ ID NO: 29), VIRDSNVVQLDV (SEQ ID NO: 30), GKNTGDHFVLYM (SEQ ID NO: 31), RLVSYSGVLFFLK (SEQ ID NO: 32), GPLPSYLQFVGI (SEQ ID NO: 33), RNRLHLSMLVRP (SEQ ID NO: 34), LVLFLNHGHFVA (SEQ ID NO: 35), AGQWHRVSVRWG (SEQ ID NO: 36), KMPYVSLELEMR (SEQ ID NO: 37), RYVVLPR (SEQ ID NO: 38), VRWGMQQIQLVV (SEQ ID NO: 39), TVFSVDQDNMLE (SEQ ID NO: 40), APMSGRSPSLVLK (SEQ ID NO: 41), VLVRVERATVFS (SEQ ID NO: 42), RNIAEIIKDI (SEQ ID NO: 43), PGRWHKVSVRWE (SEQ ID NO: 76), VLLQANDGAGEF (SEQ ID NO: 77).

In another preferred embodiment of the present invention, said growth factor binding motif derived from heparin binding domain of collagen can be a peptide of KGHRGF (SEQ ID NO: 44), TAGSCLRKFSTM (SEQ ID NO: 45), or GEFYFDLRLKGDK (SEQ ID NO: 46).

In another preferred embodiment of the present invention, said growth factor binding motif derived from heparin binding domain of vitronectin can be a peptide of KKQRFRHRNRKGYRSQ (SEQ ID NO: 47).

In another preferred embodiment of the present invention, said heparin binding motif derived from heparin binding domain of bone sialoprotein can be a peptide of KRSR (SEQ ID NO: 48), or KRRA (SEQ ID NO: 49).

In another embodiment of the present invention, growth factor binding biomolecule such as heparin, capable of binding various growth factors including basic fibroblast growth factor (bFGF), transforming growth factor β (TGF-β), or platelet derived growth factor (PDGF), may be added to said protein substrate to mimic native ECM wherein growth factors are embedded.

In another embodiment of the present invention, said growth factor binding biomolecule can be heparin and sulfated hyaluronic acid. In a preferred embodiment of the present invention, said growth factor binding biomolecule can be low molecular heparin. In another preferred embodiment of the present invention, said sulfated hyaluronic acid can be high sulfated hyaluronic acid.

In one aspect, the present invention provides an ECM mimetic protein in the form of recombinant adhesive protein comprising two domains of:
1) A positively charged mussel adhesive protein domain that interacts with a negatively charged component to form microcarrier formation:
2) A growth factor binding domain which is capable of immobilizing or sequester growth factors to activate or inhibit a cognate growth factor receptor;

According to the present invention, any recombinant mussel adhesive protein can be used for the purpose of this invention. In an embodiment of the present invention, the recombinant mussel adhesive protein may comprise, consist essentially of, or consist of the peptide of SEQ ID NOs: 1-6, and 60-74. In a preferred embodiment of the present invention, the recombinant mussel adhesive protein may be selected from foot protein 1 decapeptide repeat (SEQ ID NO: 1), foot protein 3 (SEQ ID NO: 2), foot protein 5 (SEQ ID NO: 3-4) or its combination. Preferably, the hybrid of foot protein 1 decapeptide repeat and foot protein 3, foot protein 1 decapeptide repeat and foot protein 5, or foot protein 1, foot protein 3 and foot protein 5. Preferably, the hybrid protein (SEQ ID NO: 5 and SEQ ID NO: 6) consisted of six repeats of foot protein I decapeptide at both the N-and C-termini of M. edulis foot protein 5 (SEQ ID NO: 3) or M. galloprovincialis foot protein 5 (SEQ ID NO: 4) is used for the present invention.

The GF binding domain in the present invention may be growth factor binding or syndecan binding peptide motif derived from ECM protein including collagen, fibronectin, laminin, vitronectin, fibrinogen, tenascin, or bone sialoprotein. The growth factor bound or sequestered by growth factor binding or syndecan binding motifs includes basic fibroblast growth factor (bFGF), platelet-derived growth factor (PDGF), epidermal growth factor (EGF), and vascular endothelial growth factor (VEGF), and the cytokine bound or sequestered by growth factor or syndecan binding motifs are transforming growth factor β (TGF-β), interleukin-2, and interleukin-6.

In another aspect, the present invention provides an ECM mimetic microcarrier for sustained growth factor delivery for bioprocess or tissue engineering application.

In another aspect, the invention provides a composition comprising the ECM mimetic substrate of the first aspect and a pharmaceutically-acceptable carrier for cell therapy, wound healing or tissue engineering.

In another aspect, the present invention provides a supplement as a synthetic extracellular matrix retaining growth factors or cytokines for cell culture or its related application.

In another aspect, the present invention provides a method of making ECM mimetic microcarrier for growth factor delivery to cell or tissue of interest.

According to the present invention, any negatively charged components can be used for the purpose of this invention. In an embodiment of the present invention, a negatively charged components can be a glycosaminoglycans (GAGs). GAGs also known as mucopolysaccharides, are negatively-charged polysaccharide compounds. They are composed of repeating disaccharide units that are present in every mammalian tissue. Most GAGs are first attached to the core protein and polymerized in the Golgi apparatus, where they are subsequently sulfated. Hyaluronan, a non-sulfated free polysaccharide is synthesized at the plasma membrane level (Maccioni HJ. (2007) J Neurochem. 103 Suppl 1:81-90).

In a preferred embodiment of the present invention, said GAGs may be selected from heparin, heparin analogs, low molecular weight heparin, glycosaminoglycans, hyaluronic acid, or alginate.

In a preferred embodiment of the present invention, an integrin binding motif and/or a heparin binding motif is bound to N-terminal and/or C-terminal of the recombinant adhesive protein. In another preferred embodiment of the present invention, both of an integrin binding motif and a heparin binding motif are bound to N-terminal or C-terminal of the recombinant adhesive protein.

The present invention provides an ECM mimetic microcarrier to immobilize growth factors to deliver to cells, tissues, or organs. Embodiments as well as features and advantages of the present invention will be apparent from the further descriptions herein.

### Advantageous Effects of Invention

The ECM mimetic microcarrier of the present invention may be used for cell culture related applications, for example, supplement for bioprocess for stem cell expansion, delivery of growth factor for tissue engineering, or therapeutic applications.

### BRIEF DESCRIPTION OF DRAWINGS

FIG. 1 is an illustration of the ECM-GF complex coated microcarrier according to the invention.

FIG. 2 is an illustration of the ECM mimetic microcarrier according to the invention; which comprises ECM mimetic containing peptide motif to bind growth factors and alginate as core substrate, wherein growth factors were bound by ECM mimetic peptide.

FIG. 3a and FIG. 3b represent ECM mimetic motif coated alginate microcarrier formed and suspended in PBS buffer solution, indicating the ECM mimetic motif coated microcarrier can be used as microcarrier for cell expansion. FIG. 3a represents ECM mimetic microcarrier with on average 300 μm formed in high concentration of alginate solution (1 wt %) and FIG. 3b represents ECM mimetic microcarrier with on average 800 μm formed in low concentration of alginate solution (5wt %).

FIG. 4 represents the effect of ECM mimetic microcarrier on the growth and proliferation of human foreskin fibroblast in serum condition (2% and 10% FBS).

FIG. 5 represents ECM mimetic substrate conjugated to a microcarrier via EDC/NHS chemistry, as generally known in the art.

FIG. 6 represents the effect of ECM mimetic substrate conjugated microcarrier on the growth and proliferation of murine myoblast (C2C12) in serum condition. Compared to Control (Cytodex 3, collagen coated microcarrier), ECM mimetic substrate conjugated microcarrier showed two times higher proliferation.

FIG. 7a to FIG. 7c represent the mouse myoblast grown on three different microcarriers. In comparison to commercially available microcarrier as Control (FIG. 7a), cells were grown much more on the surface of ECM mimetic substrate conjugated microcarrier (FIG. 7b and FIG. 7c, respectively) as confirmed by CCK assay results shown in FIG. 6.

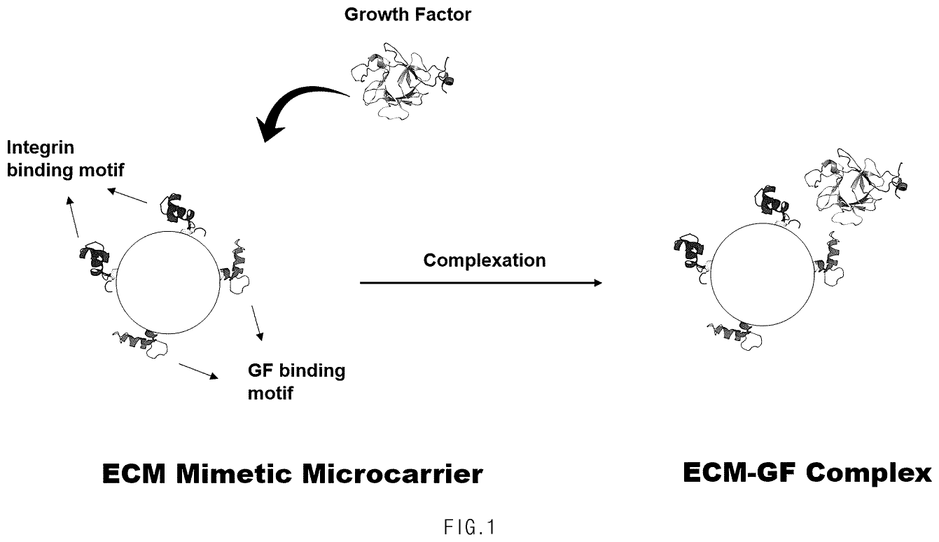

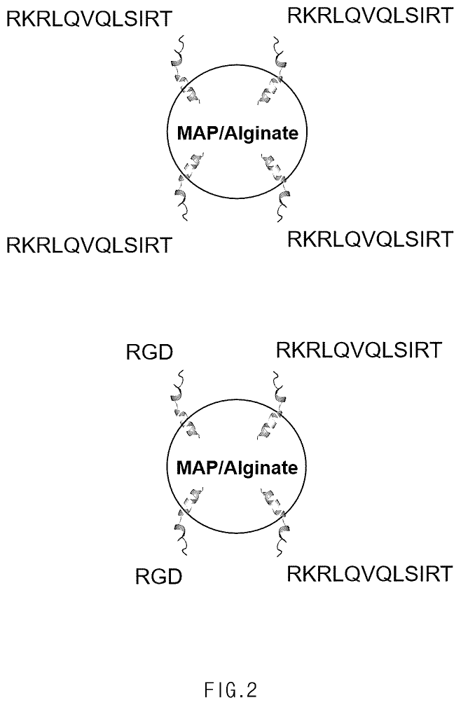

### MODE FOR THE INVENTION

The present invention is directed to an ECM mimetic microcarrier comprising a positively charged recombinant adhesive protein genetically functionalized with a growth factor binding motif which is capable of binding or sequestering growth factors and a negatively charged component to form a microcarrier complex via electric interaction.

An ECM mimetic protein substrate conjugated microcarrier can deliver one or more different growth factors, simultaneously or selectively, to expand cells or regenerate tissue. A protein substrate may be provided in the form of recombinant adhesive protein comprising three domains of:
1) A positively charged recombinant mussel adhesive protein domain that interacts with a negatively charged components, wherein said mussel adhesive protein is genetically functionalized with growth factor binding motif;
2) A growth factor binding motif which is capable of immobilizing or sequestering growth factors or cytokines to activate or inhibit a cognate growth factor receptor or cytokine receptor; and
3) An integrin binding motif which is capable of targeting specific integrin receptors.

As used herein, a recombinant mussel adhesive protein refers to a fusion protein comprising mussel foot protein FP-5 and mussel foot protein FP-1 decapeptide. In one embodiment, a protein substrate provided here comprise a mussel foot protein that is selected from the group consisting SEQ ID NOs: 5-6.

As used herein, the term “GF binding peptide motif” refers to a short peptide derived from heparin binding or syndecan binding domain of extracellular matrix proteins such as, including but not limited to, fibronectin domain III, laminin LG domain, collagen heparin binding domain, vitronectin heparin binding domain, or fibrinogen. In one embodiment, the GF binding peptide motif comprises 5-40 amino acids or its combination thereof. In various embodiments, the GF binding peptide motif comprises an amino acid sequence selected from the heparin binding or syndecan binding peptide group consisting of fibronectin-derived KYILRWRPKNS (SEQ ID NO: 7), YRVRVTPKEKTGPMKE (SEQ ID NO: 8), SPPRRARVT (SEQ ID NO: 9), ATETTITIS (SEQ ID NO: 10), VSPPRRARVTDATETTITISWRTKTETITGFG (SEQ ID NO: 11), ANGQTPIQRYIK (SEQ ID NO: 12), KPDVRSYTITG (SEQ ID NO: 13), PRARITGYIIKYEKPGSPPREVVPRPRPGV (SEQ ID NO: 14), WQPPRARI (SEQ ID NO: 15), WQPPRARITGYIIKYEKPG (SEQ ID NO: 16), YEKPGSPPREVVPRPRP (SEQ ID NO: 17), KNNQKSEPLIGRKKT (SEQ ID NO: 18), or laminin-derived GLIYYVAHQNQM (SEQ ID NO: 19), RKRLQVQLSIRT (SEQ ID NO: 20), GLLFYMARINHA (SEQ ID NO: 21), KNSFMALYLSKG (SEQ ID NO: 22), VVRDITRRGKPG (SEQ ID NO: 23), RAYFNGQSFIAS (SEQ ID NO: 24), GEKSQFSIRLKT (SEQ ID NO: 25), TLFLAHGRLVFMFNVGHKKL (SEQ ID NO: 26), TLFLAHGRLVFM (SEQ ID NO: 27), LVFMFNVGHKKL (SEQ ID NO: 28), GAAWKIKGPIYL (SEQ ID NO: 29), VIRDSNVVQLDV (SEQ ID NO: 30), GKNTGDHFVLYM (SEQ ID NO: 31), RLVSYSGVLFFLK (SEQ ID NO: 32), GPLPSYLQFVGI (SEQ ID NO: 33), RNRLHLSMLVRP (SEQ ID NO: 34), LVLFLNHGHFVA (SEQ ID NO: 35), AGQWHRVSVRWG (SEQ ID NO: 36), KMPYVSLELEMR (SEQ ID NO: 37), RYVVLPR (SEQ ID NO: 38), VRWGMQQIQLVV (SEQ ID NO: 39), TVFSVDQDNMLE (SEQ ID NO: 40), APMSGRSPSLVLK (SEQ ID NO: 41), VLVRVERATVFS (SEQ ID NO: 42), RNIAEIIKDI (SEQ ID NO: 43), PGRWHKVSVRWE (SEQ ID NO: 76), VLLQANDGAGEF (SEQ ID NO: 77) or collagen-derived KGHRGF (SEQ ID NO: 44), TAGSCLRKFSTM (SEQ ID NO: 45), GEFYFDLRLKGDK (SEQ ID NO: 46), or vitronectin-derived KKQRFRHRNRKGYRSQ (SEQ ID NO: 47), or bone sialoprotein derived KRSR (SEQ ID NO: 48), or KRRA (SEQ ID NO: 49).

As used herein, the term “microcarrier” or “microsphere,” “microbead,” “bead,” or like terms refer to a small discrete particle for use in culturing cells and to which cells can attach, grow or proliferate. Microcarriers can be in any suitable shape, such as rods, spheres, and the like. Some microcarriers have been prepared to present specific polypeptide sequences at the surface, which polypeptides provide specific interaction with integrin of the cells. Examples of such microcarriers include gelatin or collagen linked to dextran beads or to polystyrene beads. While having various advantages, such microcarriers are made of animal derived materials and are not suitable for culturing cells dedicated to cell therapies due to the risk of xenogenic contamination through, for example, pathogen proteins or viruses.

In one embodiment, the present invention discloses an ECM mimetic microcarrier that provides a GF binding peptide motif to sequester or bind to growth factors. Any suitable GF binding peptide motif can be selected from the group consisting heparin binding or syndecan binding domain derived from fibronectin domain III, laminin LG domain, collagen heparin domain, vitronectin heparin domain, fibrinogen, or bone sialoprotein as described in details in the definition term of heparin binding motif above.

In one embodiment, an ECM mimetic protein to sequester or bind to basic fibroblast growth factor (bFGF) is disclosed. Generally, the GF binding peptide motif can be selected from heparin/syndecan binding domain of fibronectin, laminin and collagen for bFGF binding. Preferably, the fibronectin-derived peptide PRARITGYIIKYEKPGSPPREVVPRPRPGV (SEQ ID NO: 14), WQPPRARI (SEQ ID NO: 15), laminin-derived peptide RYVVLPR (SEQ ID NO: 38), VRWGMQQIQLVV (SEQ ID NO: 39), VLVRVERATVFS (SEQ ID NO: 42), or collagen-derived KGHRGF (SEQ ID NO: 44) can be selected to sequester or bind to bFGF.

In another embodiment, the present invention discloses an ECM mimetic microcarrier to provide GF binding peptide motif to sequester or bind to transforming growth factor β (TGF-β). The GF binding peptide motif can be selected from heparin/syndecan binding domain of fibronectin, laminin, collagen, vitronectin, or bone sialoprotein for TGF-β binding. Preferably, fibronectin-derived motif ANGQTPIQRYIK (SEQ ID NO: 12), KPDVRSYTITG (SEQ ID NO: 13), PRARITGYIIKYEKPGSPPREVVPRPRPGV (SEQ ID NO: 14), WQPPRARI (SEQ ID NO: 15), WQPPRARITGYIIKYEKPG (SEQ ID NO: 16), YEKPGSPPREVVPRPRP (SEQ ID NO: 17), or laminin-derived peptide GLIYYVAHQNQM (SEQ ID NO: 19), RKRLQVQLSIRT (SEQ ID NO: 20), GLLFYMARINHA (SEQ ID NO: 21), KNSFMALYLSKG (SEQ ID NO: 22), VVRDITRRGKPG (SEQ ID NO: 23), RAYFNGQSFIAS (SEQ ID NO: 24), GEKSQFSIRLKT (SEQ ID NO: 25), TLFLAHGRLVFMFNVGHKKL (SEQ ID NO: 26), TLFLAHGRLVFM (SEQ ID NO: 27), LVFMFNVGHKKL (SEQ ID NO: 28), GAAWKIKGPIYL (SEQ ID NO: 29), VIRDSNVVQLDV (SEQ ID NO: 30), GKNTGDHFVLYM (SEQ ID NO: 31), RLVSYSGVLFFLK (SEQ ID NO: 32), GPLPSYLQFVGI (SEQ ID NO: 33), RNRLHLSMLVRP (SEQ ID NO: 34), LVLFLNHGHFVA (SEQ ID NO: 35), AGQWHRVSVRWG (SEQ ID NO: 36), KMPYVSLELEMR (SEQ ID NO: 37), RYVVLPR (SEQ ID NO: 38), VRWGMQQIQLVV (SEQ ID NO: 39), TVFSVDQDNMLE (SEQ ID NO: 40), APMSGRSPSLVLK (SEQ ID NO: 41), VLVRVERATVFS (SEQ ID NO: 42), RNIAEIIKDI (SEQ ID NO: 43),), PGRWHKVSVRWE (SEQ ID NO: 76), VLLQANDGAGEF (SEQ ID NO: 77), or collagen-derived KGHRGF (SEQ ID NO: 44), TAGSCLRKFSTM (SEQ ID NO: 45), GEFYFDLRLKGDK (SEQ ID NO: 46), or vitronectin-derived KKQRFRHRNRKGYRSQ (SEQ ID NO: 47), or bone sialoprotein derived KRSR (SEQ ID NO: 48), KRRA (SEQ ID NO: 49) can be selected to bind to or sequester TGF-β. More preferably, the heparin binding motif can be selected from fibronectin derived ANGQTPIQRYIK (SEQ ID NO: 12), KPDVRSYTITG (SEQ ID NO: 13), YEKPGSPPREVVPRPRP (SEQ ID NO: 17), or laminin derived KNSFMALYLSKG (SEQ ID NO: 22), RYVVLPR (SEQ ID NO: 38), GKNTGDHFVLYM (SEQ ID NO: 31), RLVSYSGVLFFLK (SEQ ID NO: 32), VLVRVERATVFS (SEQ ID NO: 42), or vitronectin derived KKQRFRHRNRKGYRSQ (SEQ ID NO: 47), or bone sialoprotein derived KRSR (SEQ ID NO: 48), or KRRA (SEQ ID NO: 49).

In another embodiment, the present invention discloses an ECM mimetic microcarrier to provide GF binding peptide motif to sequester or bind to platelet-derived growth factor (PDGF). The PDGF binding motif can be selected from laminin derived motif GLIYYVAHQNQM (SEQ ID NO: 19), RKRLQVQLSIRT (SEQ ID NO: 20), GLLFYMARINHA (SEQ ID NO: 21), KNSFMALYLSKG (SEQ ID NO: 22), VVRDITRRGKPG (SEQ ID NO: 23), RAYFNGQSFIAS (SEQ ID NO: 24), GEKSQFSIRLKT (SEQ ID NO: 25), TLFLAHGRLVFMFNVGHKKL (SEQ ID NO: 26), TLFLAHGRLVFM (SEQ ID NO: 27), LVFMFNVGHKKL (SEQ ID NO: 28), GAAWKIKGPIYL (SEQ ID NO: 29), VIRDSNVVQLDV (SEQ ID NO: 30), GKNTGDHFVLYM (SEQ ID NO: 31), RLVSYSGVLFFLK (SEQ ID NO: 32), GPLPSYLQFVGI (SEQ ID NO: 33), RNRLHLSMLVRP (SEQ ID NO: 34), LVLFLNHGHFVA (SEQ ID NO: 35), AGQWHRVSVRWG (SEQ ID NO: 36), KMPYVSLELEMR (SEQ ID NO: 37), RYVVLPR (SEQ ID NO: 38), VRWGMQQIQLVV (SEQ ID NO: 39), TVFSVDQDNMLE (SEQ ID NO: 40), APMSGRSPSLVLK (SEQ ID NO: 41), VLVRVERATVFS (SEQ ID NO: 42), RNIAEIIKDI (SEQ ID NO: 43), PGRWHKVSVRWE (SEQ ID NO: 76), VLLQANDGAGEF (SEQ ID NO: 77), or vitronectin derived KKQRFRHRNRKGYRSQ (SEQ ID NO: 47), or bone sialoprotein derived KRSR (SEQ ID NO: 48), KRRA (SEQ ID NO: 49). More preferably, the PDGF binding motif can be selected from RKRLQVQLSIRT (SEQ ID NO: 20), KNSFMALYLSKG (SEQ ID NO: 22), RYVVLPR (SEQ ID NO: 38), GKNTGDHFVLYM (SEQ ID NO: 31), VLVRVERATVFS (SEQ ID NO: 42), VLLQANDGAGEF (SEQ ID NO: 77), or vitronectin derived KKQRFRHRNRKGYRSQ (SEQ ID NO: 47), or bone sialoprotein derived KRSR (SEQ ID NO: 48).

The present invention also provides a method of making ECM mimetic microcarrier.

The present invention also provides a method of ECM mimetic microcarrier to bind or sequester growth factors.

The present invention also provides a method to stabilize growth factors or cytokines against loss of biological activity for long term in cell culture conditions by admixing a protein substrate comprising a growth factor or cytokine binding motif with growth factor or cytokine in cell culture medium or buffer solution such as PBS buffer.

In one embodiment, a composition comprising an ECM mimetic microcarrier presenting GF binding motif RKRLQVQLSIRT (SEQ ID NO: 20) or VLLQANDGAGEF (SEQ ID NO: 77) as microcarrier added to cell culture medium such as DMEM for cell growth and proliferation is provided.

Hereinafter, the present invention will be described in detail with reference to Preparation Examples, Examples, and Experimental Examples thereof.

However, it should be understood that the following Preparation Examples, Examples, and Experimental Examples are given for the purpose of illustration of the present invention only, and are not intended to limit the scope of the present invention.

### EXAMPLES

### Example 1. Preparation of an ECM Mimetic Microcarrier to Present Heparin Binding Motif

E. coli based protein expression system was commercialized to produce a variety of mussel adhesive proteins including fusion protein of mussel foot protein 1 and foot protein 5 in an efficient way (see US2020/0062809 A1 and WO2011/115420 A2), and the mussel adhesive proteins are commercially available under Trademarks MAPTrix™ marketed by Kollodis BioSciences, Inc. The method for preparation of mussel adhesive proteins is fully described in US2020/0062809 A1 and WO2011/115420A2 which is hereby incorporated by reference for all purposes as if fully set forth herein.

Two types of protein substrates having SEQ ID NO: 20 (RKRLQVQLSIRT) and SEQ ID NO: 59 (RGD) were recombinantly designed and expressed in E. coli expression system and purified as set forth in US2020/0062809 A1 and WO2011115420A2. A number of protein substrate having an ECM mimetic motif was produced with the same procedure. All protein substrate was lyophilized and stored at refrigerator for further experiment.

MAP solution (SEQ ID NO:20 and SEQ ID NO: 59) were dissolved separately in distilled water and 20 mM sodium acetate buffer (pH 6.5), respectively, producing a 0.1 mg/mL of solutions, respectively.

5 g of sodium alginate was added to 20 mL sodium acetate buffer (pH 6.5), and magnetically stirred to sodium alginate at 60° C. and dissolves fully, followed by addition of calcium chloride to form 20 mM Ca²⁺ solution.

Adding MAP solution to the sodium alginate solution with 10 mM of EDC and Sulfonated NHS to immobilize ECM mimetic MAP to sodium alginate with constant temperature magnetic agitation and reaction 1 hour at room temperature. The suction filtration product was used to obtains ECM mimetic coated alginate microcarrier.

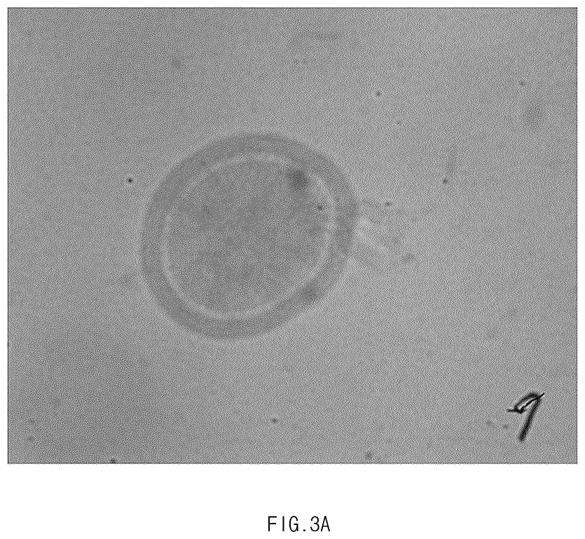

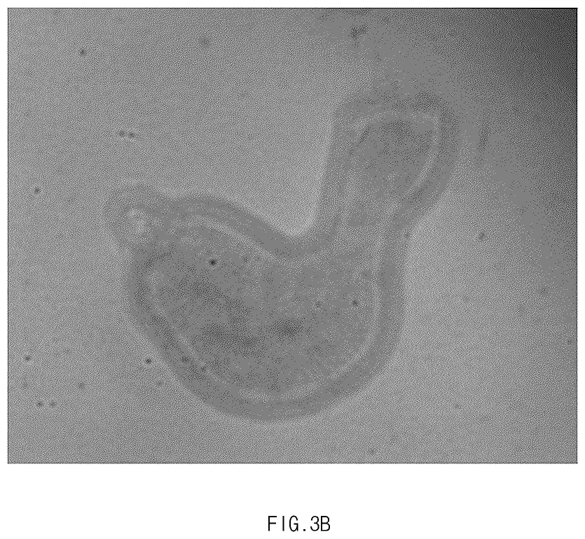

The microcarriers formed above was presented in the FIG. 3a and FIG. 3b, respectively. FIG. 3a is the microcarrier to present RKRLQVQLSIRT motif and GF binding motif RKRLQVQLSIRT and integrin binding motif RGD in FIG. 3b.

### Example 2. ECM Mimetic Microcarrier for Cell Growth

Cell growth assays were performed using human foreskin fibroblasts (Hs68, ATCC) in DMEM medium (Invitrogen) supplemented with 2% and 10% fetal bovine serum (FBS). Cells were seeded at 1,500 cells/well, followed by addition of 10 and 50 μl of ECM mimetic microcarrier solution, and then incubated for 48 h at 37° C. in CO₂ incubator. Then, CCK-8 assay was performed to determine cell growth. Hs68 cells were checked for mycoplasma contamination and used in passages from 5 to 10. The protein substrate without ECM peptide motif were used as negative and positive control, respectively.

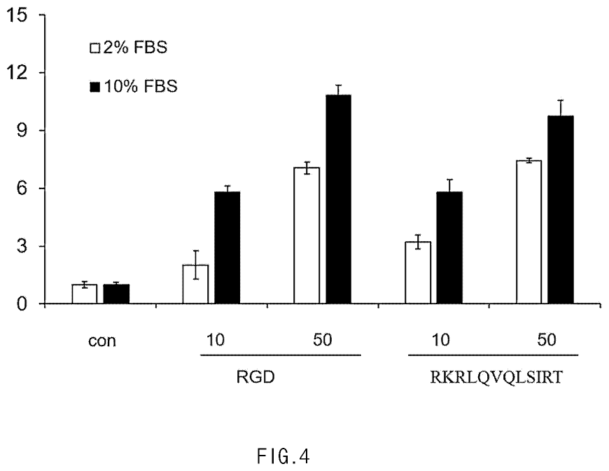

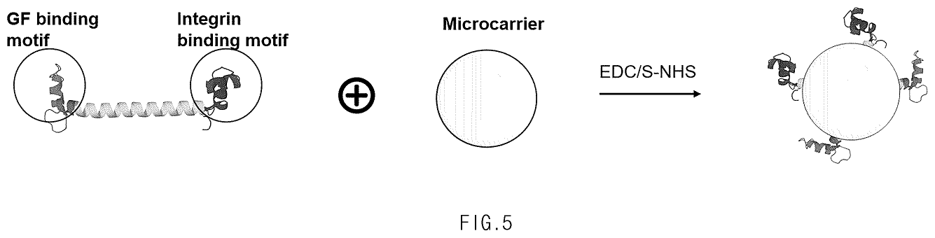

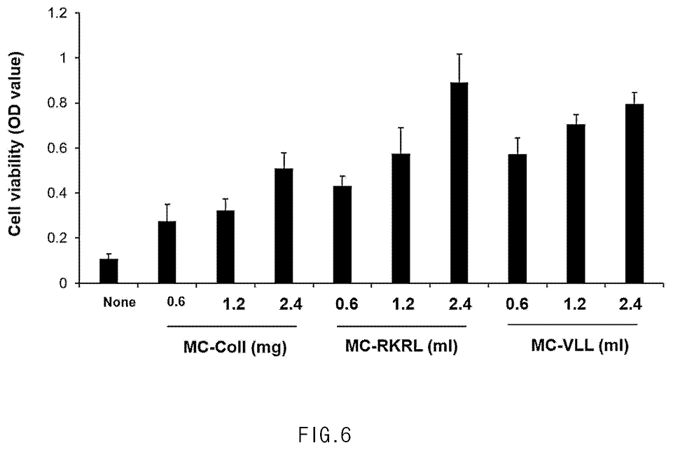

As shown in FIG. 4, both ECM mimetic motif coated microcarrier supported cell growth and proliferation. Microcarrier presenting ECM mimetic motif showed more excellent effect on cell growth and proliferation than microcarrier without ECM motif.

### Example 3. Preparation of ECM Mimetic Coated Microcarrier

15 mg of dry, denatured gelatin coated microcarrier (about 150 to 210 micrometer particle size) was added to a 10 mL of 20 mM sodium acetate solution (pH 6.5) accompanied by 1 mL solution of EDC/NHS (20 mM in DI water) and allowed to mix on a shaker for 20 minutes. The solution was aspirated, rinsed twice with 1 mL of water, aspirated, and then 10 mL of ECM mimetic substrate containing RKRLQVQLSIRT (SEQ ID NO: 20) and VLLQANDGAGEF (SEQ ID NO: 77) were added and allowed mix for 60 min. After the reaction, the solution was removed by aspiration and the microcarriers were treated with 20 mL of 70% ethanol for 30 minutes, followed by washing with DI water and dried overnight under aseptic condition.

### Example 4. The Effect of GF Binding Motif on Myoblast Cell Proliferation

C2C12 cells were purchased from ATCC (Manassas, VA) and cultured in DMEM media supplemented with 10% (v/v) fetal bovine serum and 1% (v/v) penicillin/streptomycin in a humidified incubator at 37° C. and 5% CO₂. Microcarriers (solohill, sigma) coated with collagen and adhesive protein fused with RKRLQVQLSIRT (SEQ ID NO: 20) and VLLQANDGAGEF (SEQ ID NO: 77) were suspended at a concentration of 60, 120, 240 mg/ml in a distilled water. Before cell seeding, microcarriers were added to an ultra-low attachment flat 96 well plate. Myoblasts (3,000 cells/well), C2C12, were detached from cultured dish and then seeded in ultra-low attachment 96 plate including microcarriers. As a negative control (none), myoblasts were cultured in ultra-low attachment plate without microcarriers.

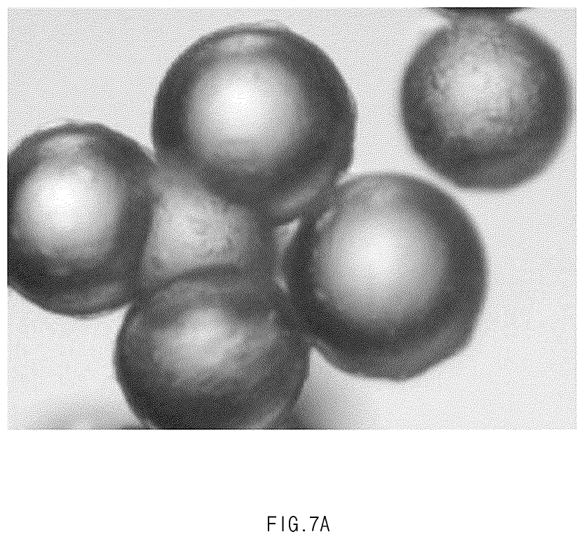

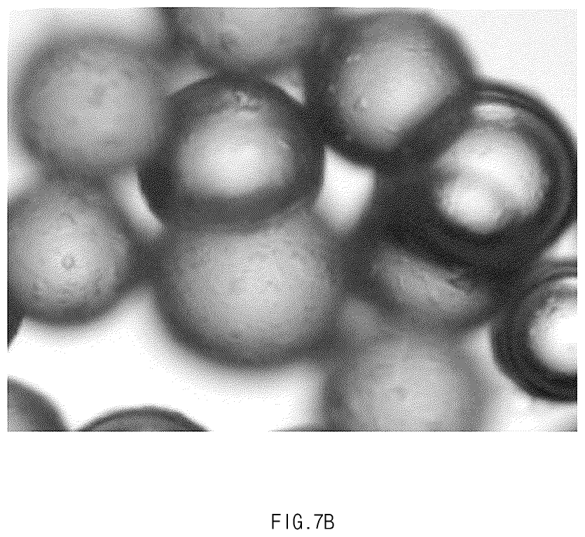

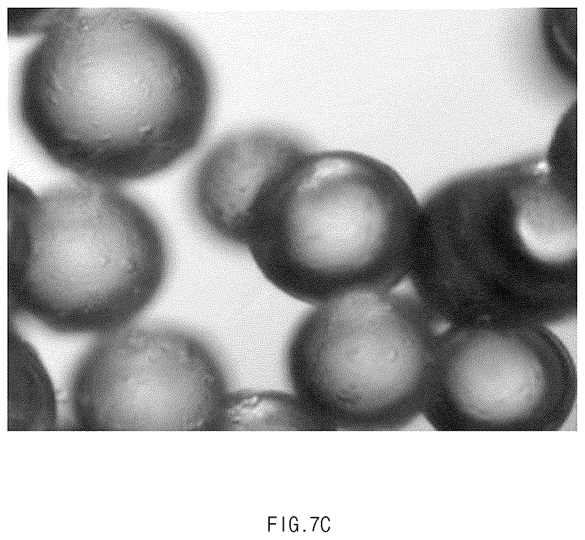

After 96 hr incubation, differential interference and phase contrast images of microcarriers were acquired and cell viability was determined by CCK-8 assay following manufacturer's instruction. As seen in FIG. 7a to FIG. 7c, the microcarriers coated with adhesive proteins fused with RKRLQVQLSIRT (SEQ ID NO: 20) and VLLQANDGAGEF (SEQ ID NO: 77) shows 175% and 155% enhancement in myoblast proliferation, respectively, as compared with the collagen coated microcarrier.

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. An ECM mimetic microcarrier comprising a positively charged recombinant adhesive protein genetically functionalized with a growth factor binding motif which is capable of binding or sequestering growth factors and a negatively charged component to form a microcarrier complex via electric interaction.

2. The ECM mimetic microcarrier of claim 1, wherein said growth factor binding motif is derived from the group consisting of fibronectin domain III, laminin globular domain, heparin binding domain of collagen, heparin binding domain of vitronectin, and heparin binding domain of bone sialoprotein.

3. The ECM mimetic microcarrier of claim 2, wherein said growth factor binding motif derived from fibronectin domain III is selected from the group consisting of KYILRWRPKNS (SEQ ID NO: 7), YRVRVTPKEKTGPMKE (SEQ ID NO: 8), SPPRRARVT (SEQ ID NO: 9), ATETTITIS (SEQ ID NO: 10), VSPPRRARVTDATETTITISWRTKTETITGFG (SEQ ID NO: 11), ANGQTPIQRYIK (SEQ ID NO: 12), KPDVRSYTITG (SEQ ID NO: 13), PRARITGYIIKYEKPGSPPREVVPRPRPGV (SEQ ID NO: 14), WQPPRARI (SEQ ID NO: 15), WQPPRARITGYIIKYEKPG (SEQ ID NO: 16), YEKPGSPPREVVPRPRP (SEQ ID NO: 17), and KNNQKSEPLIGRKKT (SEQ ID NO: 18).

4. The ECM mimetic microcarrier of claim 2, wherein said heparin binding motif derived from laminin globular domain is selected from the group consisting of GLIYYVAHQNQM (SEQ ID NO: 19), RKRLQVQLSIRT (SEQ ID NO: 20), GLLFYMARINHA (SEQ ID NO: 21), KNSFMALYLSKG (SEQ ID NO: 22), VVRDITRRGKPG (SEQ ID NO: 23), RAYFNGQSFIAS (SEQ ID NO: 24), GEKSQFSIRLKT (SEQ ID NO: 25), TLFLAHGRLVFMFNVGHKKL (SEQ ID NO: 26), TLFLAHGRLVFM (SEQ ID NO: 27), LVFMFNVGHKKL (SEQ ID NO: 28), GAAWKIKGPIYL (SEQ ID NO: 29), VIRDSNVVQLDV (SEQ ID NO: 30), GKNTGDHFVLYM (SEQ ID NO: 31), RLVSYSGVLFFLK (SEQ ID NO: 32), GPLPSYLQFVGI (SEQ ID NO: 33), RNRLHLSMLVRP (SEQ ID NO: 34), LVLFLNHGHFVA (SEQ ID NO: 35), AGQWHRVSVRWG (SEQ ID NO: 36), KMPYVSLELEMR (SEQ ID NO: 37), RYVVLPR (SEQ ID NO: 38), VRWGMQQIQLVV (SEQ ID NO: 39), TVFSVDQDNMLE (SEQ ID NO: 40), APMSGRSPSLVLK (SEQ ID NO: 41), VLVRVERATVFS (SEQ ID NO: 42), PGRWHKVSVRWE (SEQ ID NO: 76), VLLQANDGAGEF (SEQ ID NO: 77), and RNIAEIIKDI (SEQ ID NO: 43).

5. The ECM mimetic microcarrier of claim 2, wherein said growth factor binding motif derived from heparin binding domain of collagen is selected from the group consisting of KGHRGF (SEQ ID NO: 44), TAGSCLRKFSTM (SEQ ID NO: 45), and GEFYFDLRLKGDK (SEQ ID NO: 46).

6. The ECM mimetic microcarrier of claim 2, wherein said growth factor binding motif derived from is heparin binding domain of vitronectin KKQRFRHRNRKGYRSQ (SEQ ID NO: 47).

7. The ECM mimetic microcarrier of claim 2, wherein said growth factor binding motif derived from heparin binding domain of bone sialoprotein is KRSR (SEQ ID NO: 48), or KRRA (SEQ ID NO: 49).

8. The ECM mimetic microcarrier of claim 1, wherein said growth factor binding motif is capable of binding basic fibroblast growth factor (bFGF), transforming growth factor β (TGF-β), or platelet derived growth factor (PDGF).

9. The ECM mimetic microcarrier of claim 1, wherein the recombinant adhesive protein is derived from a recombinant mussel adhesive protein.

10. The ECM mimetic microcarrier of claim 1, wherein the recombinant mussel adhesive protein comprises the peptide selected from the group consisting of SEQ ID NOS: 1-6, and 60-74.

11. The ECM mimetic microcarrier of claim 1, wherein an integrin binding motif and/or a heparin binding motif is bound to N-terminal and/or C-terminal of the recombinant adhesive protein.

12. The ECM mimetic microcarrier of claim 1, wherein both of an integrin binding motif and a heparin binding motif are bound to N-terminal or C-terminal of the recombinant adhesive protein.
